(19) United States
(12) Patent Application Publication
(10) Pub. No.: US 2011/0295221 A1
(43) Pub. Date: Dec. 1, 2011

(54) STOMA SUPPORT PLATE AND HARNESS

(76) Inventor: Tim Brown, Portage, IN (US)

(21) Appl. No.: 13/115,779

(22) Filed: May 25, 2011

Related U.S. Application Data
(60) Provisional application No. 61/347,900, filed on May 25, 2010.

(51) Int. Cl. A61F 5/449

(52) U.S. Cl. 604/343

(57) ABSTRACT

A stoma support plate and harness includes a support plate including a body, an aperture and a harness fastener; and a harness including at least one plate fastener and a closure. A method of supporting an ostomy skin barrier includes the steps of: providing a support plate including a body, an aperture and a harness fastener; placing the support plate over an ostomy skin barrier such that the ostomy skin bag opening and adaptor are accessible through the support plate aperture; and securing the support plate against the ostomy skin barrier with a harness including at least one plate fastener and a closure, wherein the harness wraps and closes around the body of user to apply pressure to the ostomy skin barrier to hold the ostomy skin barrier in place, such that an ostomy bag may be secured to the ostomy skin barrier through the aperture in the support plate.

### CROSS-REFERENCE TO RELATED APPLICATIONS

This application incorporates by reference and claims priority to U.S. Provisional Patent Application No. 61/347,900 filed May 25, 2010.

### BACKGROUND OF THE INVENTION

The present subject matter relates generally to a stoma support plate and harness. More specifically, the present invention relates to a support plate and harness for providing compressive force against a stoma skin barrier to maintain its position and effectiveness.

A stoma is an opening connecting a portion of a body cavity to the environment. Stomata are sometimes created through medical procedures, for example to the esophagus, stomach, duodenum, ileum, colon, pleural cavity, ureter, bladder, renal pelvis, etc. One well-known stoma is a colostomy. A colostomy creates a stoma by drawing a portion of the large intestine or colon through an incision in the abdominal wall and suturing it into place. The stoma provides an alternative channel for feces to leave the body.

To facilitate the collection of fecal matter as it leaves the body, an ostomy skin barrier is applied to the body surrounding the stoma. An ostomy pouch, or other collection device, may then be connected to the ostomy skin barrier. The skin barrier is typically applied to the skin using an adhesive intended to hold the skin barrier in place and prevent contamination into or from the environment.

The usable life of the adhesive used in a typical skin barrier maxes out at approximately one week, though it is more common for the skin barrier to last less than four days. Hirsute men get significantly less usable life from the adhesive than women. In some cases, the adhesive will lose its effectiveness in less than twenty four hours.

Accordingly, a need exists for a device that is easy to use, cost effective and is adapted to extend the usable life of a typical stoma skin barrier.

### BRIEF SUMMARY OF THE INVENTION

The stoma support plate and harness disclosed herein is easy to use, cost effective and is adapted to extend the usable life of a typical stoma skin barrier. The stoma support plate and harness disclosed herein includes a support plate adapted to be placed between a skin barrier and an ostomy pouch and a harness for holding the support plate securely in place.

In one example, a stoma support plate and harness includes a rigid plastic ring with an opening through which an ostomy bag on one side may connect to a skin barrier on the other. A releasable fastener, such as Velcro, is used to attach a harness to the plastic ring. In this example, the harness is a pair of elastic straps that attach to the top and bottom of the support plate and extend around the torso of the user. The elastic straps provide compressive force along the support plate to maintain the support plate in position, thereby extending the usable life of the skin barrier.

For example, a stoma support plate and harness may include a support plate including a body, an aperture and a harness fastener; and a harness including at least one plate fastener and a closure. The support plate may be formed from a generally rigid material. The outer perimeter of the body may be approximately circular. The aperture may also be approximately circular. The harness fastener may include two fastening portions located on opposing sides of the aperture and each of the two fastening portions may include a fastener to secure to the harness. In one example, the fasteners are one side of a hook and loop fastening system. The harness may include at least one elastic band sized to be wrapped around the waist of a user. The harness may include two elastic bands, each sized to be wrapped around the waist of a user and further including a closure adapted to secure the corresponding elastic band around the waist of the user.

A method of supporting an ostomy skin bather may include the steps of: providing a support plate including a body, an aperture and a harness fastener; placing the support plate over an ostomy skin bather such that the ostomy skin bag opening and adaptor are accessible through the support plate aperture; and securing the support plate against the ostomy skin barrier with a harness including at least one plate fastener and a closure, wherein the harness wraps and closes around the body of user to apply pressure to the ostomy skin barrier to hold the ostomy skin bather in place, such that an ostomy bag may be secured to the ostomy skin bather through the aperture in the support plate.

An advantage of the stoma support plate and harness disclosed herein is to give the wearer the confidence to leave the house or sleep through the night without fearing a leak in the seal.

Another advantage of the stoma support plate and harness is to extend the life of the skin barrier to save money for the user.

A further advantage of the stoma support plate and harness is to provide a system for use with existing skin barriers and ostomy bags that is unobtrusive, comfortable to wear and effective.

Yet another advantage of the stoma support plate and harness is to provide a compressive, stabilizing force against a skin barrier that does not interfere with the operation of the ostomy bag.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a perspective view of a skin barrier, support plate with harness and ostomy bag.

FIG. 2 is a perspective view of the skin barrier, support plate with harness and ostomy bag shown in FIG. 1, in an assembled configuration.

### DETAILED DESCRIPTION OF THE INVENTION

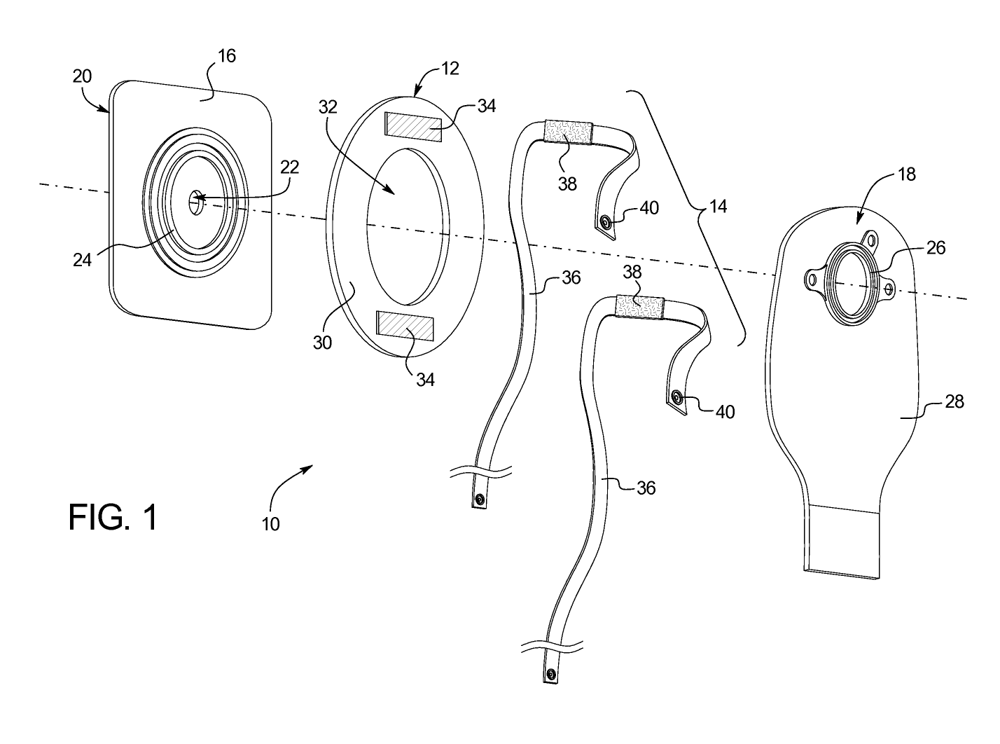

FIG. 1 illustrates an ostomy system 10 incorporating a support plate 12 and a harness 14, as disclosed herein. As shown in FIG. 1, the ostomy system 10 includes an ostomy skin barrier 16 and an ostomy bag 18, whereby the skin barrier 16 is held in place against the body of a wearer by combination of the support plate 12 and the harness 14.

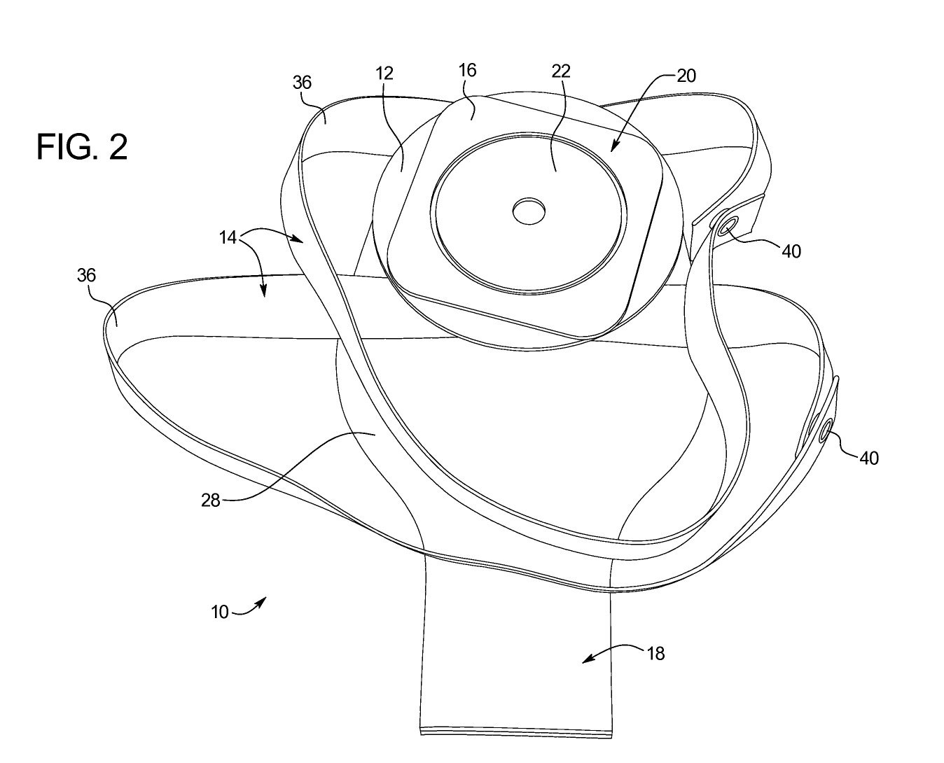

FIG. 2 illustrates the ostomy system 10 shown in FIG. 1 with the elements assembled as they would be in use. As shown, the ostomy skin barrier 16 and an ostomy bag 18 are joined together surrounding the support plate 12 and the harness 14, as described further herein.

In the example of the ostomy system 10 shown in FIGS. 1 and 2, the skin barrier 16 is a skin barrier such as the one sold under the trademark SUR-FIT Natura® Low-Pressure Adaptor by ConvaTec Inc. As shown, the skin barrier 16 includes an adhesive surface 20, an opening 22 and an adaptor 24 to which the ostomy bag 18 attaches.

As further shown in the example illustrated in FIGS. 1 and 2, the ostomy bag 18 includes a corresponding adaptor 26 and a pouch 28. The adaptors 24 and 26 connect together to form a seal between the ostomy skin barrier 16 and ostomy bag 18. Accordingly, while each of the adaptors 24 and 26 are shown as facing to the right in FIG. 1, the adapters 24 and 26 will face each other and cooperate in direct connection to form the seal between the ostomy skin bather 16 and ostomy bag 18.

The support plate 12 shown in FIGS. 1 and 2 includes a body 30, an aperture 32 and a harness fastener 34. In the example of the support plate 12 shown in FIGS. 1 and 2, the body 30 is a rigid plastic ring approximately five inches in diameter with a concentric aperture 32 approximately two and three-quarters inches in diameter. The support plate 12 shown is approximately one-sixteenth of an inch thick. The materials, sizes and proportions shown in FIGS. 1 and 2 are merely one example of a support plate 12. It is understood that the materials, sizes and proportions may be varied to appropriately match the stoma and/or corresponding ostomy skin bather 16 and an ostomy bag 18. For example, in the embodiment shown in FIGS. 1 and 2, the support plate 12 is sized to completely cover the adhesive surface 20. It is understood that the inner perimeter and/or outer perimeter of the support plate 12 may be non-circular, though the preferred embodiment is shown as circular to most directly correspond to the geometry of the ostomy skin barrier 16.

Although shown formed from a plastic material, the support plate 12 may be formed from any number of materials. However, it is important to note that the structural rigidity of the support plate 12 helps to apply an approximately even compressive force around the opening 22 of the skin bather 16. Accordingly, the material used should be of sufficient rigidity to accomplish the purposes of the subject matter described herein. Further, due to the nature of the environment in which it is to be used, there may be a benefit to using non-porous and easily sterilized materials for the support plate 12.

As described above, the support plate 12 includes an aperture 32 through which the adaptors 24 and 26 mate. Accordingly, one of the factors for determining the size and shape of the aperture is the stoma. Another factor is the size of the adaptors 24 and 26 of the ostomy skin bather 16 and an ostomy bag 18.

Because the support plate 12 is located between the skin barrier 16 and the ostomy bag 18, the support plate 12 is able to add compression to the skin barrier 16, without compressing or otherwise interfering with the operation of the ostomy bag 18.

The support plate 12 shown in FIGS. 1 and 2 includes a harness fastener 34 for attaching a harness 14 thereto. In the example shown, the harness fastener 34 includes a pair of releasable fasteners epoxied to the support plate 12. The releasable fasteners may be, for example, the hook-and-loop fasteners sold under the trademark Velcro® by Velcro Industries B.V. In FIGS. 1 and 2, the harness fastener 34 is two strips of Velcro® approximately one inch wide by two inches long. However, it is understood that the size of the Velcro® may be adapted to match the size and shape of the support plate 12. In addition, it is understood that other releasable fasteners may be used, such as, for example, buttons, clamps, clasps, clips, snaps, straps, ties, etc. It is further contemplated that in alternate versions of the support plate 12, the harness fastener 34 may be a non-releasable connection between the support plate 12 and the harness 14.

In the example shown in FIGS. 1 and 2, the harness fastener 34 includes two fastening portions located on opposing sides of the aperture 32. However, it is understood that various embodiments of the support plate 12 may include a harness fastener 34 that includes a fewer or greater number of parts. Further, the locations of the elements of the harness fastener 34 may be varied to accommodate the materials used in the harness fastener 34 and the harness 14. For example, it is contemplated that in an alternate example, the harness fastener 34 may include two separate elements located on opposing sides of the aperture 32, rather than above and below the aperture 32.

As further shown in FIGS. 1 and 2, the ostomy system 10 includes a harness 14. The harness 14 shown in FIGS. 1 and 2 includes a pair of elastic bands 36 adapted to be worn around the waist of a user. In the example shown, the harness 14 includes a pair of approximately one inch wide elastic bands 36. The elastic bands 36 may be adapted to match the size of the users. For example, for typical male adults, the elastic bands may stretch between twenty eight inches to fifty inches.

The harness 14 shown in FIGS. 1 and 2 includes a pair of elastic bands 36, each including a plate fastener 38 and a closure 40. The plate fasteners 38 are adapted to mate with the harness fastener 34 of the support plate 12. Accordingly, as shown in FIGS. 1 and 2, the plate fasteners 38 are the corresponding mating half of the hook-and-loop fasteners used in the support plate 12. The plate fasteners 38 are epoxied to the elastic bands 36. However, as described above, the plate fasteners 38 may be any type of appropriate fasteners and further may be securely attached to the elastic bands 36 in any number of ways.

Further, the elastic bands 36 shown in FIGS. 1 and 2 each include a closure 40. The closures 40 shown in FIGS. 1 and 2 are snap closures used to adapt the elastic bands 36 into belts. Accordingly, various closures may be implemented, such as, for example, buttons, clamps, clasps, clips, snaps, straps, ties, etc.

As shown, the harness 14 is formed by the two elastic bands 36 functioning as belts which fasten to the support plate 12 to hold the support plate 12 in place against the user's body. There are numerous forms in which the harness 14 may be embodied. For example, the harness may be a single piece that wraps around the body of the user that splits (i.e., forks) into two pieces that apply the pressure on either side of the support plate 12. In addition, while described and illustrated as incorporating elastic bands, the harness 14 may alternatively include any structure that helps to provide the compressive force to the support plate 12. Accordingly, any number of examples of harnesses 14 may be adapted for use with the support plate 12.

Although in description of the subject matter disclosed herein has been made primarily with reference to a stoma formed from a colostomy, it is understood that the support plate 12 and harness 14 described herein may be adapted for use with various stomata in which a material or device is to be held in place around the stoma.

It should be noted that various changes and modifications to the presently preferred embodiments described herein will be apparent to those skilled in the art. Such changes and modifications may be made without departing from the spirit and scope of the present invention and without diminishing its attendant advantages.

I claim:

1. A stoma support plate and harness comprising:
a support plate including a body, an aperture and a harness fastener; and
a harness including at least one plate fastener and a closure.

2. The stoma support plate and harness of claim 1 wherein the support plate is formed from a generally rigid material.

3. The stoma support plate and harness of claim 2 wherein the outer perimeter of the body is approximately circular.

4. The stoma support plate and harness of claim 3 wherein the aperture is approximately circular.

5. The stoma support plate and harness of claim 4 wherein the harness fastener includes two fastening portions located on opposing sides of the aperture.

6. The stoma support plate and harness of claim 5 wherein each of the two fastening portions comprises a fastener to secure to the harness.

7. The stoma support plate and harness of claim 6 wherein the fasteners are one side of a hook and loop fastening system.

8. The stoma support plate and harness of claim 1 wherein the harness includes at least one elastic band sized to be wrapped around the waist of a user.

9. The stoma support plate and harness of claim 8 wherein the harness includes two elastic bands, each sized to be wrapped around the waist of a user.

10. The stoma support plate and harness of claim 9 wherein each of the two elastic bands includes a closure adapted to secure the corresponding elastic band around the waist of the user.

11. A method of supporting an ostomy skin barrier comprising the steps of:
providing a support plate including a body, an aperture and a harness fastener;
placing the support plate over an ostomy skin barrier such that the ostomy skin bag opening and adaptor are accessible through the support plate aperture; and
securing the support plate against the ostomy skin barrier with a harness including at least one plate fastener and a closure, wherein the harness wraps and closes around the body of user to apply pressure to the ostomy skin bather to hold the ostomy skin barrier in place, such that an ostomy bag may be secured to the ostomy skin barrier through the aperture in the support plate.

12. The method of claim 11 wherein the support plate is formed from a generally rigid material.

13. The method of claim 12 wherein the outer perimeter of the body is approximately circular.

14. The method of claim 13 wherein the aperture is approximately circular.

15. The method of claim 14 wherein the harness fastener includes two fastening portions located on opposing sides of the aperture.

16. The method of claim 15 wherein each of the two fastening portions comprises a fastener to secure to the harness.

17. The method of claim 16 wherein the fasteners are one side of a hook and loop fastening system.

18. The method of claim 11 wherein the harness includes at least one elastic band sized to be wrapped around the waist of a user.

19. The method of claim 18 wherein the harness includes two elastic bands, each sized to be wrapped around the waist of a user.

20. The method of claim 19 wherein each of the two elastic bands includes a closure adapted to secure the corresponding elastic band around the waist of the user.
